(19) United States
(12) Patent Application Publication
(10) Pub. No.: US 2009/0287301 A1
(43) Pub. Date: Nov. 19, 2009

(54) Coating for medical implants

(75) Inventor: Jan Weber, Maastricht (NL)

Correspondence Address: FISH & RICHARDSON PC, P.O. BOX 1022, MINNEAPOLIS, MN, 55440-1022, US

(73) Assignee: Boston Scientific, Scimed Inc., Maple Grove, MN (US)

(21) Appl. No.: 12/121,974

(22) Filed: May 16, 2008

(51) Int. Cl. A61F 2/82

(52) U.S. Cl. 623/1.46

(57) ABSTRACT

A medical implant can include a body including a bioerodible metal and a coating overlying a surface of the bioerodible metal. The coating can include a matrix that includes a fatty acid salt and nano-particles within the matrix.

### TECHNICAL FIELD

This invention relates to a coating for medical implants, and more particularly to stents.

### BACKGROUND

A medical implant can replace, support, or act as a missing biological structure. Examples of medical implants include orthopedic implants; bioscaffolding; endoprostheses such as stents, covered stents, and stent-grafts; bone screws; and aneurism coils. Some medical implants are designed to erode under physiological conditions.

Endoprostheses are typically tubular implants that can be implanted in various passageways in a body, such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

### SUMMARY

A medical implant is described that includes a body including a bioerodible metal and a coating overlying a surface of the bioerodible metal. The coating includes a matrix that includes a fatty acid salt and nano-particles within the matrix.

The fatty acid salt can include a salt of oleic acid, arachidic acid, stearic acid, palmitic acid, erucic acid, arachidonic acid, linoleic acid, linolenic acid, eicorapentacnoic acid, or a combination thereof. In some embodiments, the fatty acid salt comprises an oleic acid salt. For example, the fatty acid salt can be sodium oleate.

The nano-particles can include a metal, a ceramic, a conductive polymer, a nanoclay, or a combination thereof. In some embodiments, the nano-particles can include gold, silicone carbide, zirconium dioxide, aluminium oxide, an organic metal polyaniline, a polythiophene, a polypyrrole, montmorillonite, or a combination thereof. For example, the coating can be a coating can include gold nano-particles within a matrix of sodium oleate. The nano-particles can have an average diameter of between about 5 and 30 nm.

The bioerodible metal can include iron, magnesium, or an alloy thereof. In some embodiments, the bioerodible metal can include a rough surface. The rough surface, as the term is used herein, will have a Ra range of between 0.2 micrometers and 5 micrometers. The rough surface, in some embodiments, can be a microporous surface. The microporous surface can have pores having an average diameter in the range of 100 nanometers to 5 micrometers. The microporous surface can have a density between 0.9 and 0.2 times a density of a non-porous portion of the body.

In some embodiments, the medical implant can be an endoprosthesis (e.g., a stent).

In another aspect, an endoprosthesis includes a metal body and a coating overlying a surface of the metal body. The coating includes an oleic acid salt (e.g., sodium oleate).

In some embodiments, the coating includes nano-particles in a matrix comprising the oleic acid salt. The nano-particles can include a metal, a ceramic, a conductive polymer, a nanoclay, or a combination thereof. In some embodiments, the nano-particles can include gold, silicone carbide, zirconium dioxide, aluminium oxide, an organic metal polyaniline, a polythiophene, a polypyrrole, montmorillonite, or a combination thereof. For example, the coating can be a coating can include gold nano-particles within a matrix of sodium oleate. The nano-particles can have an average diameter of between about 5 and 30 nm.

In some embodiments, the metal body can include a bioerodible metal. For example, the bioerodible metal can include iron, magnesium, or an alloy thereof. In some embodiments, the metal body can include a rough surface. The rough surface, in some embodiments, can be a microporous surface. The microporous surface can have pores having an average diameter in the range of 100 nanometers to 5 micrometers. The microporous surface can have a density between 0.9 and 0.2 times a density of a non-porous portion of the body.

In some embodiments, the endoprosthesis is a stent.

The details of one or more embodiments are set forth in the accompanying drawings and the description below. Other features, objects, and advantages will be apparent from the description and drawings, and from the claims.

### DESCRIPTION OF DRAWINGS

FIG. 1 is a perspective view of an example of an expanded stent.

Like reference symbols in the various drawings indicate like elements.

### DETAILED DESCRIPTION

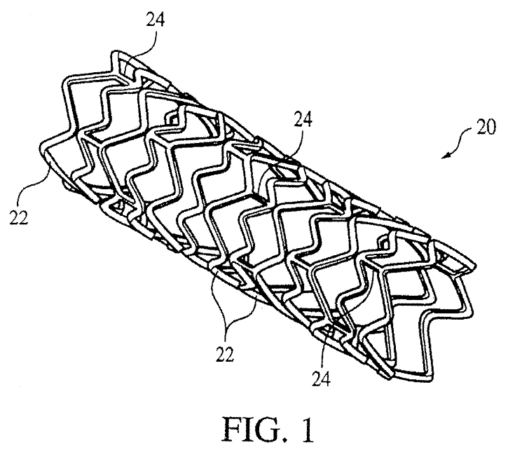

The medical implant includes a metal body and a coating including a fatty acid salt overlying a surface of the metal body. A stent 20, shown in FIG. 1, is discussed below as an example of one medical implant according to the instant disclosure. Other examples of medical implants can include orthopedic implants; bioscaffolding; bone screws; aneurism coils, heart valves; implant filters; and other endoprostheses such as covered stents and stent-grafts.

As shown in FIG. 1, stent 20 can have the form of a tubular member defined by a plurality of bands 22 and a plurality of connectors 24 that extend between and connect adjacent bands. During use, bands 22 can be expanded from an initial, small diameter to a larger diameter to contact stent 20 against a wall of a vessel, thereby maintaining the patency of the vessel. Connectors 24 can provide stent 20 with flexibility and conformability that allow the stent to adapt to the contours of the vessel.

Stent 20 can include a metal body and a coating overlying a surface of the metal body. The coating includes a fatty acid salt. The fatty acid can be, for example, a salt of oleic acid, arachidic acid, stearic acid, palmitic acid, erucic acid, arachidonic acid, linoleic acid, linolenic acid, eicorapentacnoic acid, or a combination thereof.

The fatty acid salt can include, for example, sodium, potassium, iron, and combinations thereof. For example, the fatty acid salt can be sodium oleate, potassium oleate, iron oleate, or a combination thereof. The coating of a fatty acid salt (such as oleic acid) can inhibit endothelial activation. For example, upon implantation of the stent within a physiological environment, a coating of sodium oleate can ionize to produce oleic acid, which can decrease endothelial activation.

The coating can also include nano-particles within a matrix of the salt of the fatty acid. The nano-particles can include metals, ceramics, conductive polymers, and/or nanoclays. For example, the nano-particles can include metals, such as gold. In other embodiments, the nano-particles can include ceramics such as silicone carbide, zirconium dioxide, and aluminium oxide. Examples of conductive polymers that can be included in the nano-particles include organic metal polyanilines, polythiophene, and polypyrrole. Examples of nanoclays that can be included in the nanoparticles include montmorillonite. The nano-particles can, in some embodiments, have an average diameter of between 5 nm and 30 nm. For example, gold nano-particles can have an average diameter of 12 nm. Nano-particles in the coating can improve endothelial cell coverage. For example, having nano-particles acting as nano-pillars at the surface of the coating can allow for endothelial cells to be more mobile. While nano-pillars having a height of about 13 nm can allow for endothelial cell migration, other surface nano-structures, such as hills and dimples of various sizes, can stimulate endothelial cell coverage.

The metal body of stent 20 can be bioerodible. Examples of bioerodible metals include iron, magnesium, zinc, tungsten, and alloys thereof. The coating can overlie a surface of the bioerodible metal. The coating can decrease the erosion rate of the bioerodible metal. For example, a coating of a sodium oleate by itself on an iron stent can increase the charge transfer resistance by a factor of about two. A coating of sodium oleate including gold nano-particles on an iron stent can increase the charge transfer resistance by a factor of about four. The increase in charge transfer resistance can temporarily delay the erosion of the bioerodible metal.

The metal body of stent 20 can, in some embodiments, include a stainless steel, a platinum enhanced stainless steel, a cobalt-chromium alloy, and/or a nickel-titanium alloy.

The metal body can, in some embodiments, include a rough surface. The coating can cover the rough surface. The rough surface, as the term is used herein, will have a Rₐ range of between 0.2 micrometers and 5 micrometers. For example, the surface of the metal body can include micro-pores. The micropores can have an average diameter of between 100 nanometers and 5 micrometers. The density of the micro-porous surface can have a density that is between 0.9 and 0.2 times a density of a non-porous portion of the metal body. For example, a microporous surface can be produced in an iron surface by irradiating the surface with noble ions (e.g., argon) at a dose of at least about 1×10¹⁷ ions/cm² at 300 C using Ion Beam Assisted Deposition (“IBAD”) or Plasma Immersion Ion Implantation (“PIII”). The energy range of the ions can be between 5 keV and 40 keV. A rough surface can also be produced by chemically etching the surface (e.g., with hydrofluoric acid). After roughening the surface, the coating can be applied. A rough surface can improve the adhesion of the coating to the metal body. In some embodiments, a rough surface can accelerate the erosion of a bioerodible metal body after the oleic acid salt ionizes to expose the surface of the bioerodible metal body.

A coating including a matrix of sodium oleate with gold nano-particles can, for example, be produced by mixing together HAuCl₄, NaBH₄, and sodium oleate. During the mixing process, gold nano-particles precipitate. For example, twenty milliliters of 0.001 M aqueous solution of HAuCl₄ can be added to thirty milliliters of a 0.004 M aqueous solution of NaBH₄ including 0.00025 M of sodium oleate, stirred at about 0° C. to obtain a red clear suspension/solution of sodium oleate and gold nano-particles. The precipitated gold nano-particles can have an average diameter of about 12 nm. The stent can then be coated by dipping the stent into the solution/suspension of sodium oleate and gold nano-particles. The stent can also be coated by other fluid dispensing methods such as spraying. In some embodiments, the sodium oleate and/or gold nano-particles can penetrate into micro-pores and/or grooves in a roughened surface of the medical implant. In some embodiments, prior to applying the coating to the metal body, the metal body can be cleaned by immersing the stent in a 0.2 M solution of hydrochloric acid for about 15 seconds, followed by a distilled water rinse, to produce an active surface. The coating can also be formed and applied by other methods.

The stent can, in some embodiments, include a therapeutic agent. In some embodiments, a therapeutic agent can be incorporated into a matrix of the salt of the fatty acid. In some embodiments, a therapeutic agent can be deposited over the coating and/or within a bioerodible portion of the stent. The term “therapeutic agent” includes one or more “therapeutic agents” or “drugs.” The terms “therapeutic agents” and “drugs” are used interchangeably and include pharmaceutically active compounds, nucleic acids with and without carrier vectors such as lipids, compacting agents (such as histones), viruses (such as adenovirus, adeno-associated virus, retrovirus, lentivirus and a-virus), polymers, antibiotics, hyaluronic acid, gene therapies, proteins, cells, stem cells and the like, or combinations thereof, with or without targeting sequences. The delivery mediated is formulated as needed to maintain cell function and viability. A common example of a therapeutic agent includes Paclitaxel.

Stent 20 can be of any desired shape and size (e.g., superficial femoral artery stents, coronary stents, aortic stents, peripheral vascular stents, gastrointestinal stents, urology stents, and neurology stents). Depending on the application, the stent can have a diameter of between, for example, 1 mm to 46 mm. In certain embodiments, a coronary stent can have an expanded diameter of from 2 mm to 6 mm. In some embodiments, a peripheral stent can have an expanded diameter of from 5 mm to 24 mm. In certain embodiments, a gastrointestinal and/or urology stent can have an expanded diameter of from 6 mm to about 30 mm. In some embodiments, a neurology stent can have an expanded diameter of from about 1 mm to about 12 mm. An Abdominal Aortic Aneurysm (AAA) stent and a Thoracic Aortic Aneurysm (TAA) stent can have a diameter from about 20 mm to about 46 mm.

In use, a stent can be used, e.g., delivered and expanded, using a catheter delivery system. Catheter systems are described in, for example, Wang U.S. Pat. No. 5,195,969, Hamlin U.S. Pat. No. 5,270,086, and Raeder-Devens, U.S. Pat. No. 6,726,712. Stents and stent delivery are also exemplified by the Sentinol® system, available from Boston Scientific Scimed, Maple Grove, Minn.

In some embodiments, stents can also be a part of a covered stent or a stent-graft. In other embodiments, a stent can include and/or be attached to a biocompatible, non-porous or semi-porous polymer matrix made of polytetrafluoroethylene (PTFE), expanded PTFE, polyethylene, urethane, or polypropylene.

In some embodiments, medical implants other than stents include orthopedic implants; bioscaffolding; bone screws; aneurism coils; heart valves; implant filters; and other endoprostheses such as covered stents and stent-grafts. These medical implants can be formed of a bioerodible metal and include a coating including a matrix of a fatty acid salt having metallic nano-particles within the matrix.

Other embodiments are within the claims.

What is claimed is:

1. A medical implant comprising:
a body comprising a bioerodible metal; and
a coating overlying a surface of the bioerodible metal, the coating comprising:
a matrix that includes a fatty acid salt; and
nano-particles within the matrix.

2. The medical implant of claim 1, wherein the fatty acid salt comprises a salt of oleic acid, arachidic acid, stearic acid, palmitic acid, erucic acid, arachidonic acid, linoleic acid, linolenic acid, eicorapentacnoic acid, or a combination thereof.

3. The medical implant of claim 1, wherein the fatty acid salt comprises an oleic acid salt.

4. The medical implant of claim 3, wherein the oleic acid salt comprises sodium oleate.

5. The medical implant of claim 1, wherein the nano-particles comprise a metal, a ceramic, a conductive polymer, a nanoclay, or a combination thereof.

6. The medical implant of claim 4, wherein the nano-particles comprise gold, silicone carbide, zirconium dioxide, aluminium oxide, an organic metal polyaniline, a polythiophene, a polypyrrole, montmorillonite, or a combination thereof.

7. The medical implant of claim 1, wherein the nano-particles comprise a gold.

8. The medical implant of claim 1, wherein the metallic nano-particles have an average diameter of between about 5 and 30 nm.

9. The medial device of claim 1, wherein the bioerodible metal comprises iron or an alloy thereof.

10. The medical implant of claim 1, wherein the bioerodible metal comprises magnesium or an alloy thereof.

11. The medical implant of claim 1, wherein the surface of the bioerodible metal comprises a rough surface.

12. The medical implant of claim 11, wherein the rough surface is a microporous surface having pores having an average diameter in the range of 100 nanometers to 5 micrometers.

13. The medical implant of claim 11, wherein the rough surface is a microporous surface having a density between 0.9 and 0.2 times a density of a non-porous portion of the body.

14. The medical implant of claim 1, wherein the medical implant is an endoprosthesis.

15. The medical implant of claim 1, wherein the medical implant is a stent.

16. An endoprosthesis comprising:
a metal body; and
a coating overlying a surface of the metal body, the coating comprising an oleic acid salt.

17. The endoprosthesis of claim 16, wherein the oleic acid salt comprises sodium oleate.

18. The endoprosthesis of claim 16, wherein the coating comprises nano-particles in a matrix comprising the oleic acid salt.

19. The endoprosthesis of claim 18, wherein the nano-particles comprise a metal, a ceramic, a conductive polymer, a nanoclay, or a combination thereof.

20. The endoprosthesis of claim 18, wherein the nano-particles comprise gold, silicone carbide, zirconium dioxide, aluminium oxide, an organic metal polyaniline, a polythiophene, a polypyrrole, montmorillonite, or a combination thereof.

21. The endoprosthesis of claim 18, wherein the nano-particles comprise a gold.

22. The endoprosthesis of claim 18, wherein the nano-particles have an average diameter of between about 5 and 30 nm.

23. The endoprosthesis of claim 16, wherein the metal body comprises a bioerodible metal.

24. The endoprosthesis of claim 23, wherein the bioerodible metal comprises iron or an alloy thereof.

25. The endoprosthesis of claim 23, wherein the bioerodible metal comprises magnesium or an alloy thereof.

26. The endoprosthesis of claim 16, wherein the surface of the metal body comprises a rough surface.

27. The endoprosthesis of claim 26, wherein the rough surface is a microporous surface having pores having an average diameter in the range of 100 nanometers to 5 micrometers.

28. The endoprosthesis of claim 26, wherein the rough surface is a microporous surface having a density between 0.9 and 0.2 times a density of a non-porous portion of the body.

29. The endoprosthesis of claim 16, wherein the endoprosthesis is a stent.
